(19) United States
(12) Patent Application Publication
(10) Pub. No.: US 2014/0156023 A1
(43) Pub. Date: Jun. 5, 2014

(54) METHODS OF TREATMENT EMPLOYING RETRIEVABLE ESOPHAGEAL STENT SYSTEMS

(71) Applicant: THE METHODIST HOSPITAL RESEARCH INSTITUTE, Houston, TX (US)

(72) Inventor: Shanda Haley Blackmon, Houston, TX (US)

(73) Assignee: The Methodist Hospital Research Institute, Houston, TX (US)

(21) Appl. No.: 13/801,219

(22) Filed: Mar. 13, 2013

Related U.S. Application Data
(62) Division of application No. 13/705,002, filed on Dec. 4, 2012.

(51) Int. Cl. A61F 2/04

(52) U.S. Cl. CPC A61F 2/04
USPC 623/23.65; 623/23.68

(57) ABSTRACT

A stent system is provided that includes a stent and a migration-prevention bridle extending from the stent and secured to a portion of the body so as to resist or prevent movement of the stent relative to the portion of the body. Methods of use include positioning a stent that includes a one-way valve in an unexpanded orientation in an passageway of a patient, expanding the stent after being located adjacent an esophageal perforation such that the one-way valve controls the flow of material through the esophageal passageway, and extending a migration-prevention bridle into the esophageal passageway and securing it to the stent. A second portion of the migration-prevention bridle is then secured to a body part of the patient to help resist or prevent migration of the stent. Additional methods, systems, stent apparatuses, and a kit including a stent and instructions for the use of same are also provided.

### CROSS-REFERENCE TO RELATED APPLICATIONS

The present application claims priority to U.S. patent application Ser. No. 13/705,002, attorney docket no. 37182.107, filed Dec. 4, 2012, the contents of which is specifically incorporated herein in its entirety by express reference thereto.

### STATEMENT REGARDING FEDERALLY SPONSORED RESEARCH OR DEVELOPMENT

Not Applicable.

### NAMES OF THE PARTIES TO A JOINT RESEARCH AGREEMENT

Not Applicable.

### BACKGROUND OF THE INVENTION

### Field of the Invention

The present disclosure relates generally to medical devices and in particular, stent systems. More particularly, the invention concerns systems and methods for positioning a stent (including those having one or more valves) within the body of an animal, as well as methods and systems for inhibiting, minimizing, and/or preventing movement or migration of the stent within or about the body. Also disclosed are methods for subsequently retrieving and removing the stent from the body.

### BRIEF DESCRIPTION OF THE DRAWINGS

For promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates. The following drawings form part of the present specification and are included to demonstrate certain aspects of the present invention. The invention may be better understood by reference to the following description taken in conjunction with the accompanying drawings, in which like reference numerals identify like elements, and in which:

FIG. 1 is a side view illustrating a stent according to an embodiment of the invention;

FIG. 2 is a partial cross-sectional view illustrating a patient in need of a stent according to an exemplary embodiment of the invention;

FIG. 3A is a flow chart illustrating a method for placing a stent in a patient and removing the stent from the patient according to an exemplary embodiment of the invention;

FIG. 3B is a cross-sectional view illustrating a perforation in an esophagus in the patient of FIG. 2 according to an exemplary embodiment of the invention;

FIG. 3C is a cross-sectional view illustrating the stent of FIG. 1 being positioned in the esophagus of FIG. 3B according to an exemplary embodiment of the invention;

FIG. 3D is a cross-sectional view illustrating the stent of FIG. 1 positioned in the esophagus of FIG. 3B according to an exemplary embodiment of the invention;

FIG. 3E is a cross-sectional view illustrating a migration prevention member being coupled to the stent FIG. 3D according to an exemplary embodiment of the invention;

FIG. 3F is a cross-sectional view illustrating the migration-prevention member coupled to the stent FIG. 3D according to an exemplary embodiment of the invention;

FIG. 3G is a cross-sectional view illustrating the migration prevention member secured to prevent migration of the stent of FIG. 3D in a human according to an embodiment of the invention;

FIG. 3H is a front view illustrating the migration-prevention member secured to prevent migration of the stent of FIG. 3D according to an exemplary embodiment of the invention;

FIG. 3I is a cross-sectional view illustrating the stent of FIG. 3D being removed from the human esophagus according to an exemplary embodiment of the invention;

FIG. 3J is a cross-sectional view illustrating an illustrative embodiment of the stent of FIG. 3D being removed from the human esophagus according to the invention;

FIG. 4 is a cross-sectional view illustrating a migration-prevention member secured to prevent migration of the stent of FIG. 3D according to an exemplary embodiment of the invention; and

FIG. 5 is a partial cross-sectional view illustrating the stent of FIG. 1 being used to treat an anastomosis according to an exemplary embodiment of the invention.

### DESCRIPTION OF ILLUSTRATIVE EMBODIMENTS

In the drawings and description that follows, the drawings are not necessarily to scale. Certain features of the present disclosure may be shown exaggerated in scale or in somewhat schematic form and some details of conventional elements may not be shown in the interest of clarity and conciseness. The present disclosure is susceptible to embodiments of different forms. Specific embodiments are described in detail and are shown in the drawings, with the understanding that are to be considered an exemplification of the principles of the present disclosure, and is not intended to limit the present disclosure to that illustrated and described herein. It is to be fully recognized that the different teachings of the embodiments discussed below may be employed separately or in any suitable combination to produce desired results. The various characteristics mentioned above, as well as other features and characteristics described in more detail below, will be readily apparent to those of ordinary skill in the art upon reading the following detailed description of the embodiments, and by referring to the accompanying drawings.

Stents such as, for example, esophageal stents, colorectal stents, vascular stents, biliary stents, gastric stents, anastomotic stents, and/or a variety of other stents known in the art, may be used to keep a blocked passageway open, repair leaks or perforations, treat non-perforating lesions such as strictures, dysphagia, fistulas, cancers, insults and other injuries, and/or for the treatment of a variety of other stent-repairable abnormalities known in the art. Such stents may include self-expanding stents that are positioned in a body passageway adjacent the stent-repairable abnormality, and then expanded into contact with the body part to open up the blocked passageway, cover the leak or perforation, and/or otherwise treat strictures, dysphagia, fistulas, cancers, insults and other injuries. In many cases, however, these stents tend to migrate through the body passageway such that they no longer hold open the blocked passageway at the relevant location, cover the leak or perforation, or otherwise treat the strictures, dysphagia, fistulas, cancers, insults and other injuries. Such migration can lead to a number of issues, including the failure to remedy the stent-repairable abnormality and the need to retrieve the stent after it has migrated. For example, leakage from esophageal and gastric perforation carries a high morbidity and often requires surgical intervention. In spite of promising early results from esophageal stenting, stent migration continues to plague the procedure, occurring approximately 20-60% of the time. Conventional solutions to stent migration have been to provide one or more uncovered metal portions on the stent (e.g., the entire stent, the end portions of the stent, etc.) that engage the wall of the body passageway to resist migration. Such conventional solutions, however, do not always work and conventional stents have been found to migrate with regularity.

Furthermore, in some conventional embodiments, stents may be used in body passageways of body parts that include mechanisms for opening and closing the body passageway (e.g., in the esophageal passageway adjacent an esophageal sphincter) such that the stent may inhibit or even prevent the normal closure of such body passageway. In such situations, the expanded stent may allow material to undesirably travel through the body passageway (e.g., allowing acids or other materials from the stomach to travel up through the esophagus).

Additionally, in some embodiments, it may be desirable to provide stents in a body passageway temporarily and then remove those stents from the body passageway when the stent has repaired the stent-repairable abnormality to the point where the stent is no longer needed and the body part is capable of healing itself further without stent treatment.

The present disclosure describes several embodiments of a stent system and method that reliably inhibits or prevents the migration of a stent relative to the body part and stent-repairable abnormality which it is placed adjacent to, includes a valve to inhibit or prevent material from traveling through the stent in one direction, and provides for retrieval of the stent from the body passageway. In one embodiment, a method for preventing stent migration includes securing a migration-prevention bridle to a valved stent once it has been positioned in the body part adjacent the stent-repairable abnormality. That migration-prevention bridle is then secured to another portion of the body such as, for example, the nose through the esophageal passageway and the nasal passageways, such that any movement of the stent relative to the body part and stent-repairable abnormality will be resisted (i.e., inhibited) and prevented by the migration-prevention bridle. The stent further includes a retrieval loop that may be grasped to reduce the diameter of the stent to allow the stent to be more easily removed from the body passageway relative to conventional stents.

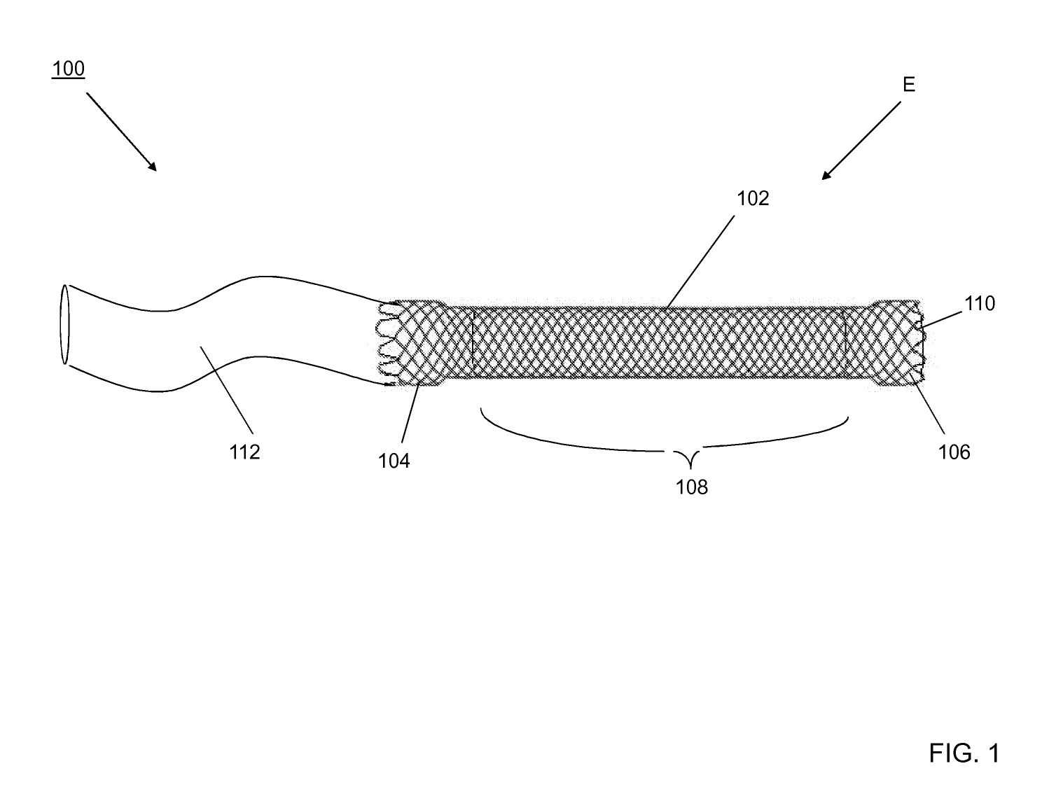

Referring initially to FIG. 1, an embodiment of a stent 100 is illustrated. The stent 100 is illustrated and described below as a partially covered, self-expanding metal stent. Those of skill in the art will recognize, however, that uncovered metal stents, plastic stents, silicone stents, polymer mesh stents, absorbable stents, and/or a variety of other stents known in the art may fall within the scope of the present disclosure. Such stents may optionally be coated with any of a number of materials, including, without limitation, those described in U.S. Pat. No. 8,034,361 directed to nanoparticular coatings on stents; U.S. Patent Appl. Publ. No. US 2004/0215336 directed to plasticizers in polymer-coated stents; and U.S. Pat. No. 6,258,121 directed to controlled-release polymer stent coatings, each of which is specifically incorporated herein in its entirety by express reference thereto. The stents of the invention may further optionally comprise or contain one or more biopolymers or coatings, and may further optionally include one or more growth factors, antibiotics, and/or additional pharmaceutical compounds to assist in healing of the tissue, etc. The stent 100 illustrated in FIG. 1 is in an expanded orientation E and includes a self-expanding mesh base 102 having a first diameter. A first end portion 104 extends from a first end of the base 102 and includes a second diameter that is larger than the first diameter. A second end portion 106 extends from a second end of the base 102 opposite the first end portion 104 and includes a third diameter that is preferably also larger than the first diameter. In some embodiments, the second diameter of the first end portion 104 is approximately equal to the third diameter of the second end portion 106. A cover 108 may extend along a majority of the base 102. The stent 100 includes a retrieval loop 110 that is operatively and, preferably physically, coupled to the mesh, or integrally formed with the mesh, of the second end portion 106 of the stent 100. Thus, the retrieval loop 110 provides a “purse-string” function that allows a force provided on the retrieval loop 110, in a direction that is along the longitudinal axis of the stent 100 or otherwise away from the stent 100, to reduce the diameter of the second end portion 106 of the stent 100, as discussed in further detail below. The stent 100 also preferably includes a one-way valve 112 that extends from the first end portion 104 of the stent 100 and that is operable to allow material to pass through the stent 100 in a first direction in its expanded orientation E, while inhibiting, or preferably entirely preventing, materials from passing through that stent 100 in a direction that is opposite to the first direction.

In an embodiment, the one-way valve 112 is a collapsible tubular member that may include a plastic material, a silicon material, and/or a variety of other materials or combinations thereof known to one of ordinary skill in the art as being suitable for implantation into a patient for an extended treatment period. In an embodiment, the one-way valve 112 may extend from the cover 108 and include the same or compatible material as the cover 108. In another embodiment, the one-way valve 112 may be coupled to the mesh of the base 102 separately from the cover 108 (e.g., an end of the one-way valve 112 may be sewn or otherwise coupled to the mesh base 102.) While a specific one-way valve has been illustrated and described, one of ordinary skill in the art will recognize that a variety of other one-way valve members that operate to restrict material travel through the stent 100 in one direction may be included on the stent 100 without departing from the scope of the present disclosure.

In an embodiment, the self-expanding mesh base 102 includes a metal material, and the cover 108 includes a non-metal material such as at least one plastic material, polymer material, or silicon material, or any combination thereof. Typically, this is a nitinol substance that is self-expanding. In an embodiment, the stent 100 may be a commercially available stent such as the Polyflex® esophageal stent available from Boston Scientific, the Ultraflex® esophageal or colonic stent available from Boston Scientific, the Wallflex® esophageal stent available from Boston Scientific, Alimaxx-ES® esophageal stent available from Merit Medical Systems (formerly Alveolus) (South Jordan, Utah, USA), or any other commercially-available stent known to one of ordinary skill in the art.

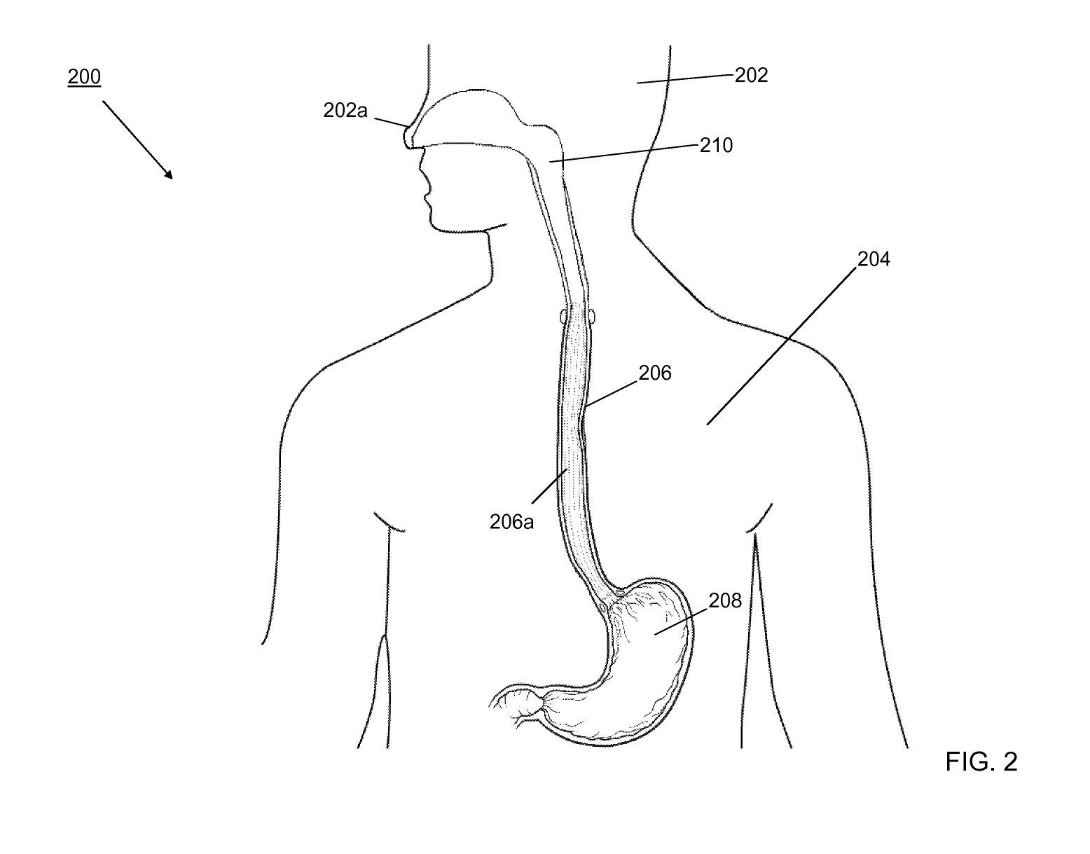

Referring now to FIG. 2, an embodiment of a patient 200 in need of stent therapy according to the invention is illustrated. The patient 200 depicted includes a head 202 extending from a torso 204 and having a nose 202a. In the illustrated embodiment, a body passageway in the patient 200 is illustrated and described below as an esophagus 206 that defines an esophageal passageway 206a and that is located in the torso 204. One of ordinary skill in the art will recognize, however, that a variety of other body passageways such as, for example, the stomach, the bowel (small and/or large), the colon, or any other body passageways defined anywhere in the gastrointestinal system and/or a variety of other body passageways known to one of ordinary skill in the art will fall within the scope of the present disclosure. The esophageal passageway 206a extends between a stomach 208 and a nasal passageway 210 that is connected to the nose 202a. One of ordinary skill in the art will recognize that intermediate passageways between the esophageal passageway 206a, the nasal passageway 210, and the nose 202a exist, but are not called out in the present disclosure to simplify the discussion herein.

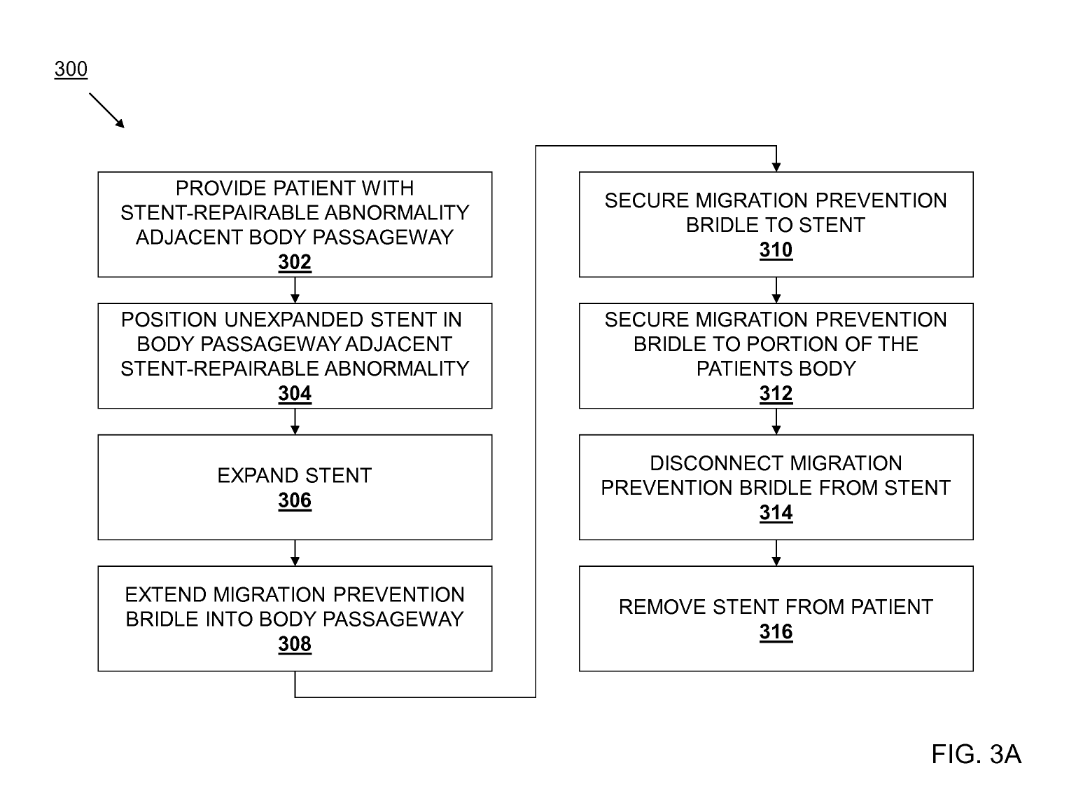

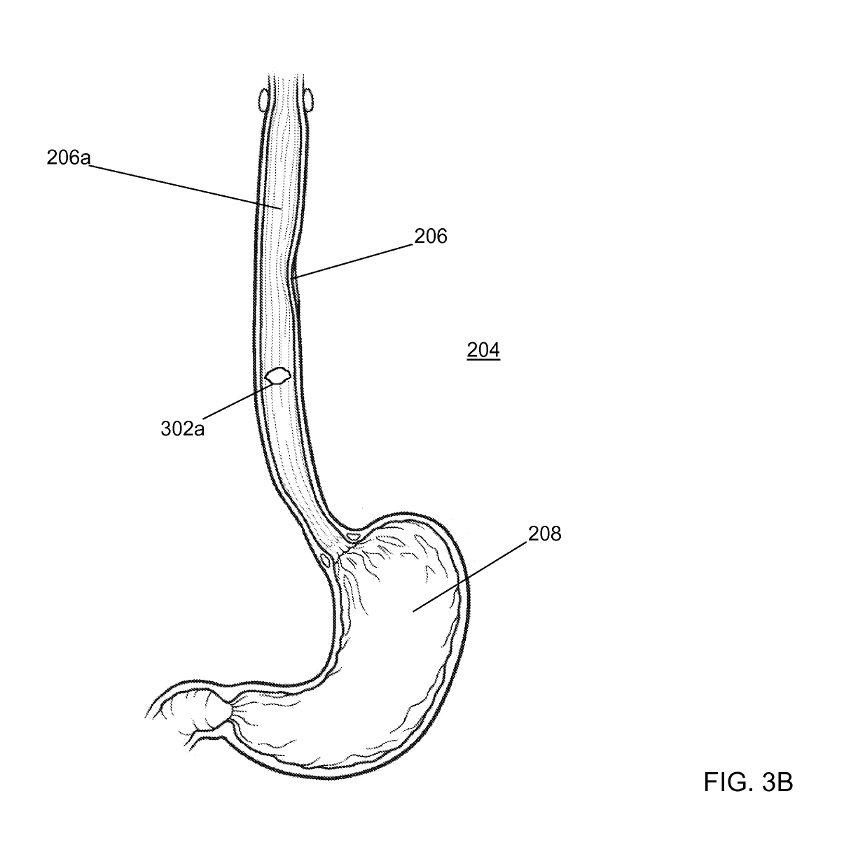

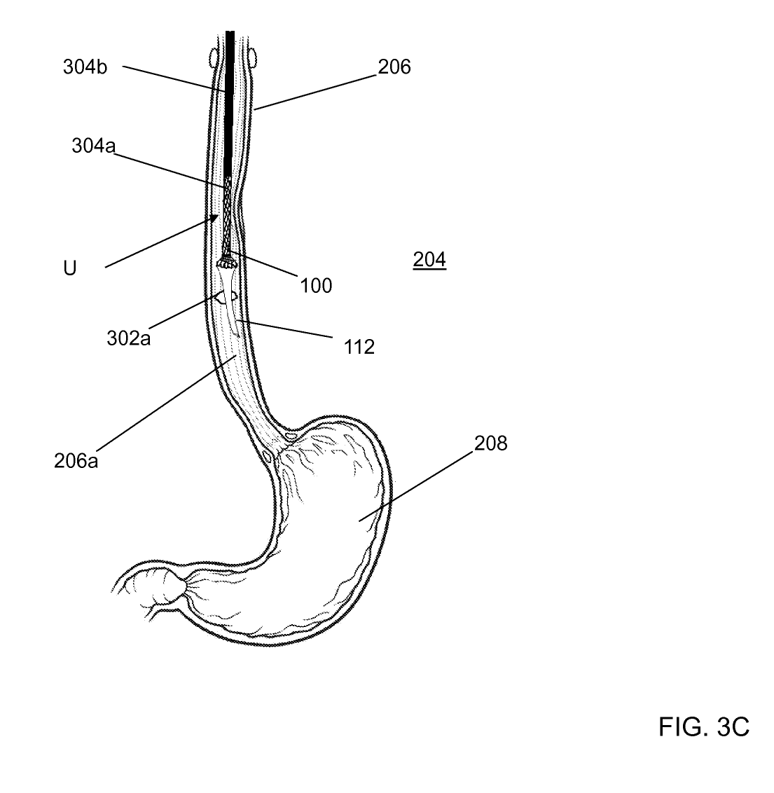

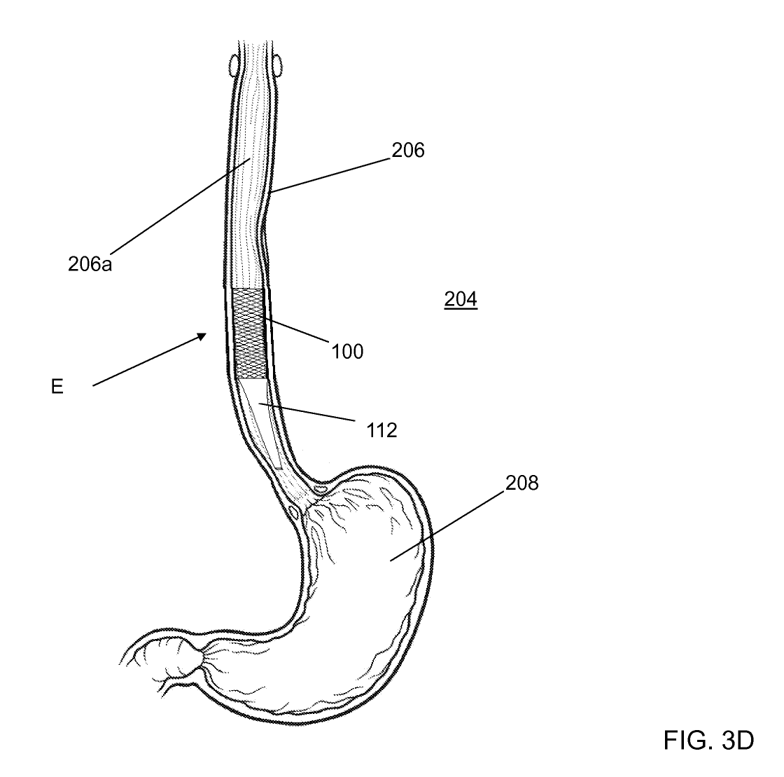

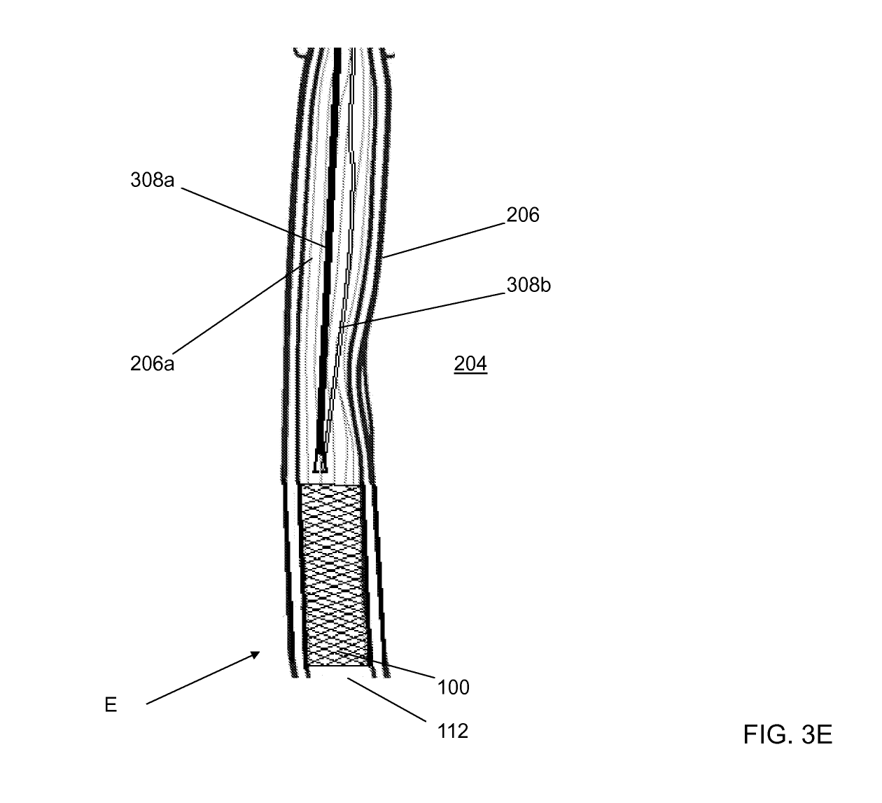

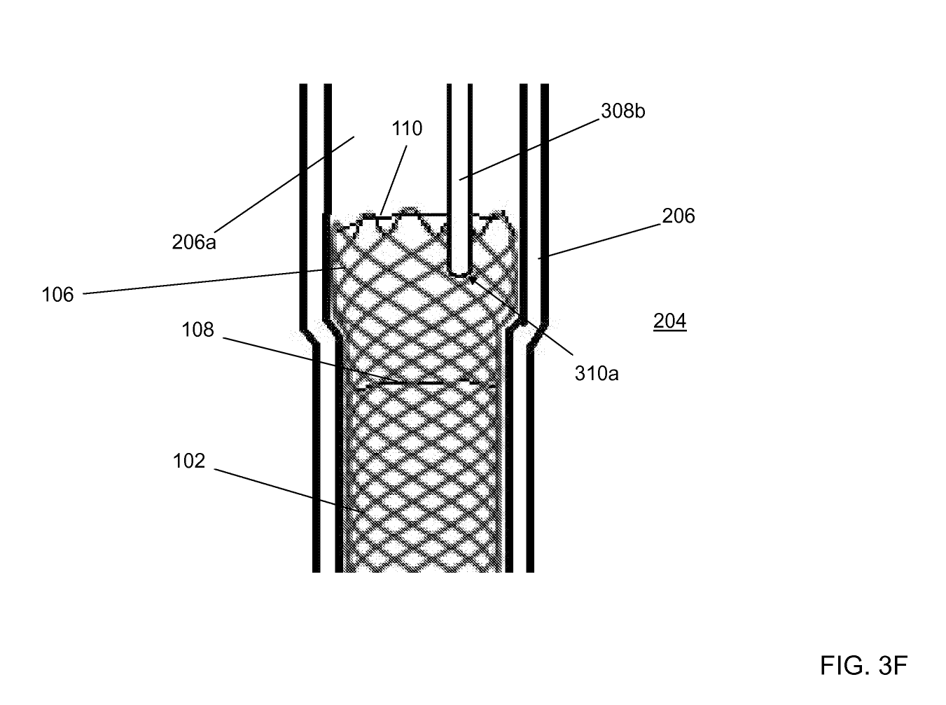

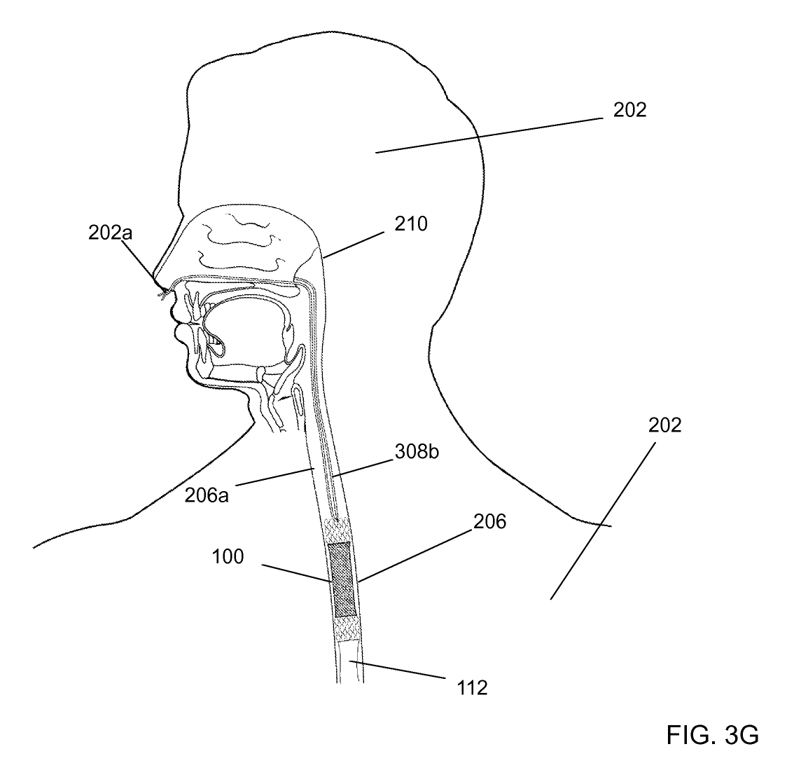

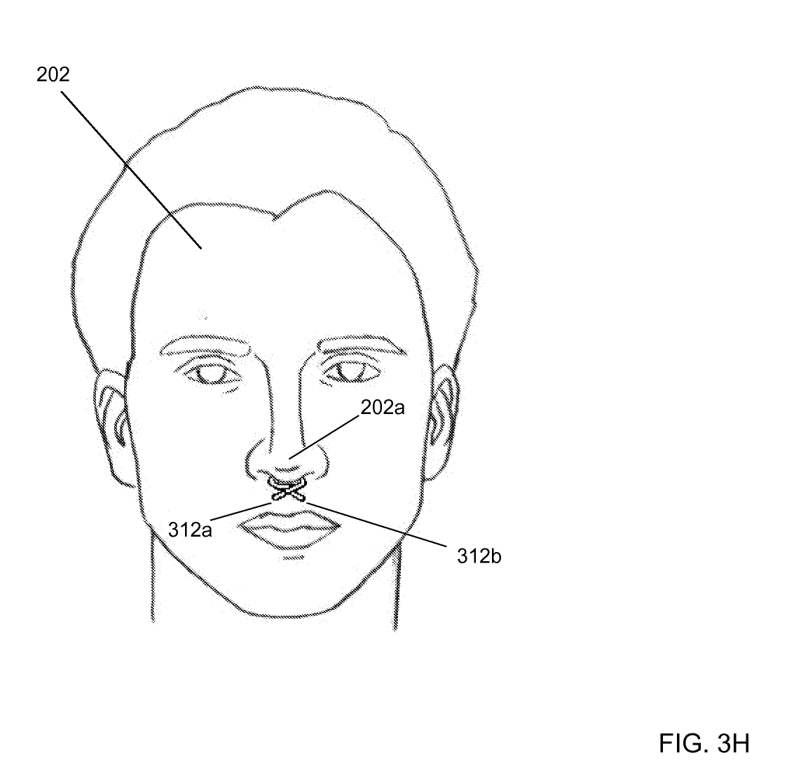

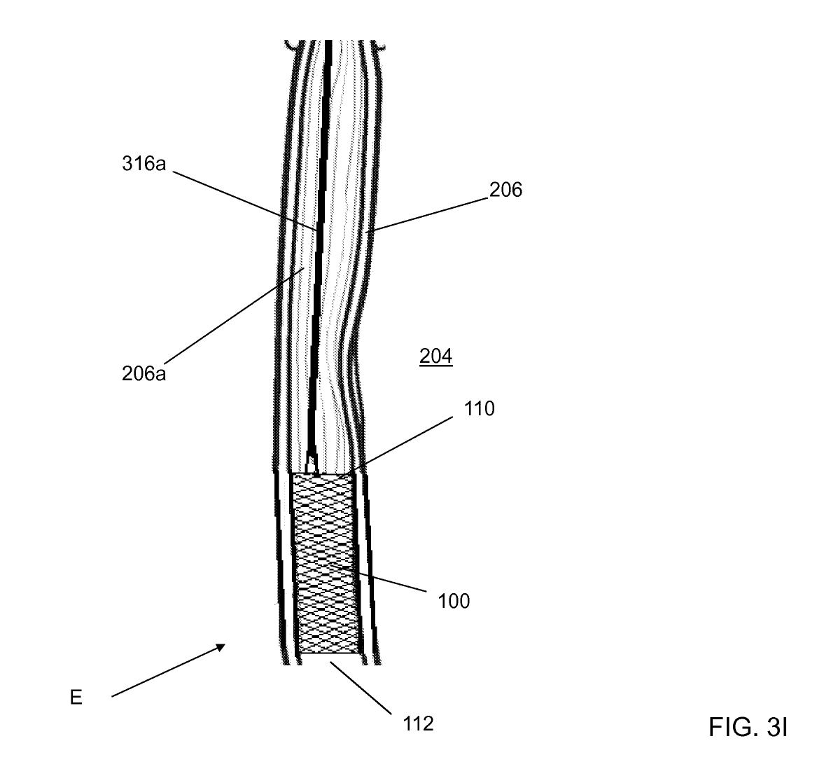

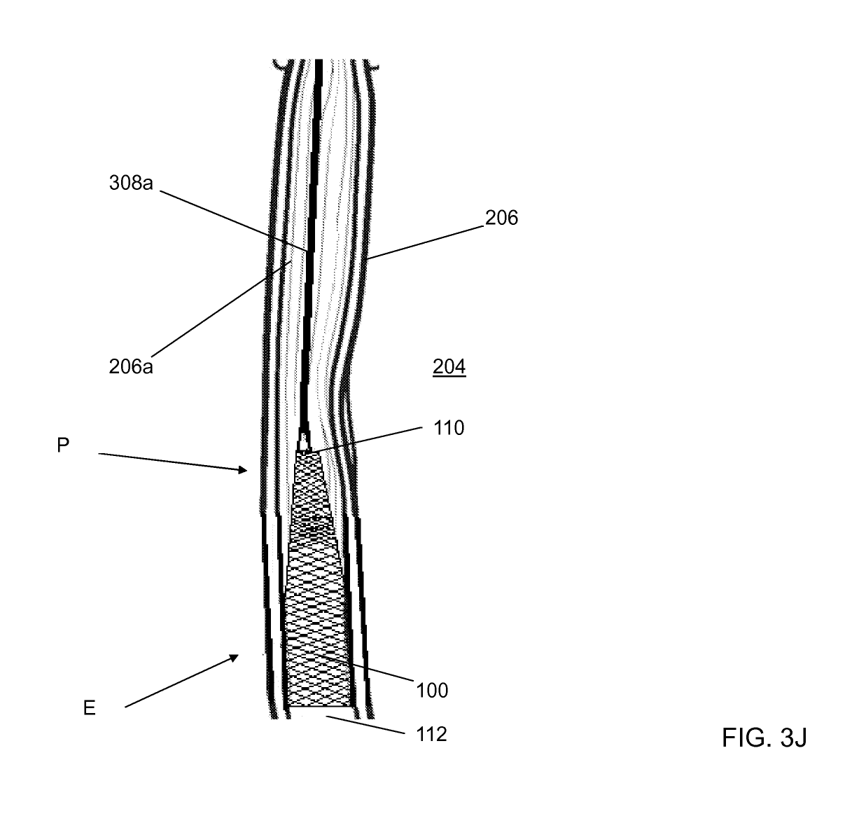

Referring now to FIG. 2, FIG. 3A, and FIG. 3B, a method 300 for placing a stent in a patient and removing the stent from the patient is illustrated. The method 300 is illustrated and described below as being performed with a stent being used to cover and/or treat a perforation in an esophagus. One of skill in the art will understand, however, that the stent systems and methods discussed below may be practiced in other passageways of the body (e.g., anywhere in the gastrointestinal system) while remaining within the scope of the present disclosure. The method 300 begins at block 302 where a patient having a stent-repairable abnormality that is located adjacent a body passageway is provided. In an embodiment, the patient 200 as illustrated in FIG. 2 is provided. As can be seen, the patient 200 includes a body passageway and has a stent-repairable abnormality that, in the embodiment illustrated in FIG. 3B, includes the esophagus 206 defining the esophageal passageway 206a and an esophageal perforation 302a that is located adjacent the esophageal passageway 206a. In other embodiments, the stent-repairable abnormality may include one or more of anastomotic leaks, benign strictures, caustic injury, aortoesophageal fistulae, spontaneous perforations, intrathoracic leaks, leaks associated with morbid obesity surgery, esophageal dilatation, non-perforating lesion such as stricture, dysphagia, fistulaes, injury, insult, and cancer, and/or a variety of other stent-repairable abnormalities known to one of ordinary skill in the art.

Referring now to FIG. 1, FIG. 3A, and FIG. 3C, the method 300 then proceeds to block 304 where a stent in an unexpanded orientation is positioned in the body passageway adjacent one or more stent-repairable abnormalities. In an embodiment, the stent 100 is placed in an unexpanded orientation U (e.g., by providing opposing forces on the first end portion 104 and the second end portion 106 of the stent) and positioned in a delivery tube 304a of a stent delivery system 304b to hold the stent 100 in the unexpanded orientation U for trans-oral delivery into the esophageal passageway 206a. In the illustrated embodiment, the stent 100 in the unexpanded orientation U is positioned in the delivery tube 304a with the one-way valve 112 extending from the stent 100 and the delivery tube 304a. In other embodiments, however, the one-way valve 112 may be positioned in the delivery tube 304a or other portions of the stent delivery system 304b during block 304 of the method 300. In an embodiment, the stent delivery system 304b selected may be one of a variety of stent delivery systems provided by Boston Scientific for use in delivering the Polyflex® esophageal stent, the Ultraflex® esophageal or colonic stent, and/or the Wallflex® esophageal stent, discussed above. In other embodiments, the stent delivery system 304b may be an endoscope. In yet other embodiments, a guide wire may be provided that extends into the body passageway for help in placement of the stent 100. As can be seen in FIG. 3C, the stent delivery system 304b may be used to move the stent 100 in the unexpanded orientation U through the esophageal passageway 206a until the stent 100 is positioned adjacent the esophageal perforation 302a.

Referring now to FIG. 1, FIG. 3A, FIG. 3C, and FIG. 3D, the method 300 then proceeds to block 306 where the stent is expanded. In an embodiment, the stent 100 may be expanded to the expanded orientation E by using the stent delivery system 304b to push the stent 100 out of the delivery tube 304a when the stent 100 is properly positioned adjacent the esophageal perforation 302a. Removal of the stent 100 from the delivery tube 304a, for example, allows the stent 100 to self-expand into contact with the wall of the esophagus 206 that defines the esophageal passageway 206a, as illustrated in FIG. 3D. Furthermore, as can be seen in FIG. 3D, the one-way valve 112 extends from the stent 100 and through the esophageal passageway 206a towards the stomach 208. Following the positioning and expansion of the stent 100 at block 306 of the method 300, the stent delivery system 304b may be removed from the esophageal passageway 206a and the patient 200. While a single stent 100 has been illustrated and described as being expanded in the esophageal passageway 206a adjacent the stent-repairable abnormality 302a, multiple stents may be positioned at least partially in the esophageal passageway 206a, including at least a portion of one stent expanded within the passageway defined by another expanded stent.

Referring now to FIG. 1, FIG. 3A, FIG. 3D, and FIG. 3E, the method 300 then proceeds to block 308 where a migration-prevention bridle is extended into the body passageway. In an embodiment, a migration-prevention bridle delivery device 308a such as, for example, endoscopic rat-tooth forceps may be used to trans-orally position a migration-prevention bridle 308b in the esophageal passageway 206a. For example, the migration-prevention bridle 308b may include a first end and a second end located opposite the migration-prevention bridle 308b from the first end. The first end may be left external to the body of the patient 200, and the migration-prevention bridle delivery device 308a may be used to grasp the second end of the migration-prevention bridle 308b and extend the migration-prevention bridle 308b from outside of the body of the patient 200 into the esophageal passageway 206a (e.g., through the mouth of the patient 200). In an embodiment, the migration-prevention bridle 308b may include umbilical tape, one or more staples, one or more sutures, tying two portions of the bridle together, and/or a variety of other materials known to one of ordinary skill in the art, including, for example without limitation endoscopic clips, including those of the Ovesco clipping device, “resolution” clips (e.g., Boston Scientific), T-fasteners (e.g., Kimberly Clark), and such like to secure the tape/stent to the wall of the esophagus. In an embodiment, at block 308 of the method 300, the migration-prevention bridle 308b (e.g., the second end discussed above) is positioned in the passageway defined by the stent 100 in its expanded orientation E and then released by the migration-prevention bridle delivery device 308a. The migration-prevention bridle thus inhibits, or preferably entirely prevents, any significant movement of the stent relative to the esophagus after the stent is properly located in the patient's esophageal passageway.

Referring now to FIG. 1, FIG. 3A, FIG. 3E, and FIG. 3F, the method 300 then proceeds to block 310 where the migration-prevention bridle is secured to the stent. In an embodiment, with the migration-prevention bridle 308b positioned in the passageway defined by the stent 100 in its expanded orientation E, the migration-prevention bridle delivery device 308a is used to thread the migration-prevention bridle 308b through an opening 310a in the mesh that makes up the second end portion 106 of the stent 100. For example, rat-tooth forceps may be passed through the mesh to grasp the second end of the migration-prevention bridle 308b in order to pull the second end of the migration-prevention bridle 308b through the mesh. As can be seen in FIG. 3F, the migration-prevention bridle 308b has been threaded through the opening 310a in the portion of the mesh that provides the second end portion 106 of the stent 100 that is uncovered by the cover 108. In this embodiment, the opening 310a is spaced apart from the retrieval loop 110 such that forces applied to the migration-prevention bridle 308b do not apply a force to the retrieval loop 110 that would cause “purse stringing” of the second end portion 106 of the stent 100, discussed in further detail herein.

Referring now to FIG. 1, FIG. 3A, FIG. 3G, and FIG. 3H, the method 300 then proceeds to block 312 where the migration-prevention bridle is secured to a portion of the patient's body. In an embodiment, once the migration-prevention bridle 308b has been secured to the stent 100 at block 310 of the method 300 by threading the migration-prevention bridle 308b through the stent 100, the migration-prevention bridle delivery device 308a may pull the second end of the migration-prevention bridle 308b back up through the esophageal passageway 206a and out of the mouth of the patient 200 such that each of the first end and the second end of the migration-prevention bridle 308b extend out of the mouth of the patient 200 while a portion of the migration-prevention bridle 308b is threaded through the stent 100. An endotracheal (ET) suction catheter may then be used to guide the first end (e.g., the first end 312a illustrated in FIG. 3H) and the second end (e.g., the second end 312b illustrated in FIG. 3H) of the migration-prevention bridle 308b through the nasal passageway 210 such that the first end of the migration-prevention bridle 308b extends out of a first nostril defined by the nose 202a and the second end of the migration-prevention bridle 308b extends out of a second nostril defined by the nose 202a. The first end and the second end of the migration-prevention bridle 308a may then be tied, as illustrated in FIG. 3G and FIG. 3H, in order to secure the migration-prevention bridle 308a to the nose 202a of the patient 200. As discussed herein, one of ordinary skill in the art may select other alternative or additional methods to tying so as to help secure the migration-prevention bridle 308b to inhibit or prevent stent movement while implanted in the patient. With the migration-prevention bridle 308a secured to the stent 100 and the nose 202a of the patient 200, any movement of the stent 100 relative to the esophagus 206 and the stent-repairable abnormality 302a is resisted or prevented by the migration-prevention bridle 308. In fact, in illustrative embodiments where the migration-prevention bridle 308a was secured to the stent 100 and the nose 202a of the patient 200, migration of the stent 100 was found to have been completely eliminated movement of the implanted stent (see e.g., David et al., Am. J. Surg., 202(6):796-801, 2011).

Furthermore, with the stent 100 secured in position relative to the esophagus 206 as discussed above, the one-way valve 112 will inhibit, or preferably prevent, any material from traveling up through the esophageal passageway 206a. For example, material such as food or liquids consumed by the patient may travel down through the esophageal passageway 206a, through the stent 100, and then through the one-way valve 112 and into the stomach 208. However, if material from the stomach 208 attempts to travel up the esophageal passageway 206a, the one-way valve 112 will collapse, fold, or otherwise deform in the esophageal passageway 206a and/or the stent 100 such that the material will be inhibited or blocked from moving through the stent 100 and up the esophageal passageway 206a. While the use of a collapsible tubular member as the one-way valve 112 is illustrated and described, one of ordinary skill in the art will recognize that a variety of other one-way valves may be used in place of, or in combination with, the one-way valve 112 illustrated in the drawings while still remaining within the scope of the present disclosure.

Referring now to FIG. 1, FIG. 3A, FIG. 3G, and FIG. 3H, the method 300 then proceeds to block 314 where the migration-prevention bridle is disconnected from the stent. In some embodiments, the migration-prevention bridle 308a may be secured to the stent 100 and the nose 202a of the patient 200 until a desired amount of tissue in-growth to the stent 100 has occurred. As discussed herein, one or more conventional stent coatings may be employed to enhance or facilitate tissue in-growth to minimize the time needed between stent implantation and release of the connection between the migration-prevention bridle 308a and the implanted stent. For example, in illustrative embodiments, it has been found that after approximately four days, tissue in-growth to the stent will occur that is sufficient to inhibit or preferably prevent migration of the stent 100 relative to the esophagus 206 and the stent-repairable abnormality 302a. Thus, at block 314 of the method 300, the first end 312a and the second end 312b of the migration-prevention bridle 308a may be untied from each other about the nose 202a, and the first end 312a of the migration-prevention bridle 308a may be pulled away from the nose 202a of the patient such that the migration-prevention bridle 308a moves through the opening 310a on the stent 100 in which it is threaded until the second end 312b of the migration-prevention bridle 308a moves through the opening 310a on the stent 100 and then may move through the esophageal passageway 206a and the nasal passageway 210 until it exits the body of the patient 200 through the nose 202a.

Referring now to FIG. 1, FIG. 3A, FIG. 3F, FIG. 3G, FIG. 3I, and FIG. 3J, in some embodiments, the method 300 may proceed to block 316 where the stent is removed from the patient. In embodiments where the stent 100 is used to repair the esophageal perforation 302a in the esophagus 206 (or other stent-repairable abnormality), once the stent-repairable abnormality has been determined to be sufficiently repaired, the stent 100 may be removed from the selected passageway, depicted as the esophageal passageway 206a. For example, following the disconnecting of the migration-prevention bridle 308b from the stent 100 in block 314 of the method 300, a stent-retrieval device 316a such as, for example, endoscopic rat-tooth forceps may be positioned in the esophageal passageway 206a adjacent the stent 100. The stent retrieval device 316a may then be used to grasp the retrieval loop 110 on the second end portion 106 of the stent 100, as illustrated in FIG. 3I. The stent retrieval device 316a is then pulled away from the stent 100 to provide a force on the retrieval loop 110 that results in a “purse string” function P that reduces the diameter of the second end portion 106 of the stent 100, as illustrated in FIG. 3J, and allows the stent 100 to be moved relative to the esophagus 206 such that the stent 100 may be removed from the body of the patient 200. In some embodiments, the migration-prevention bridle 308b may be secured to the retrieval loop 110 on the stent at block 310 of the method 300. In such embodiments, block 314 may be skipped, and the migration-prevention bridle 308b may be utilized at block 316 to remove the stent 100 from the patient 200 by, for example, pulling on the migration-prevention bridle 308a to provide a force on the retrieval loop 110 such that the “purse string” function P allows the stent 100 to be removed from the body of the patient 200.

In one embodiment, a kit including at least a stent, migration-prevention bridle, and instructions as to the use of the same, may be packaged together.

Thus, a stent apparatus, a stent system, methods of using such stents and stent systems, and kits including such stents and stent systems, have been described for positioning a stent in a body passageway of a patient that includes a one-way valve that typically only allows material to move in one direction through the stent, inhibiting or preferably preventing migration of the stent relative to the body passageway, and removing the stent from the body passageway. The systems and methods include a novel bridling technique that bridles a stent with a novel one-way valve to a body part of the patient and has been found, in experimental embodiments, to inhibit or preferably prevent movement of the stent relative to its desired position. Furthermore, that stent is provided with a means for easily retrieving the stent from the body of the patient.

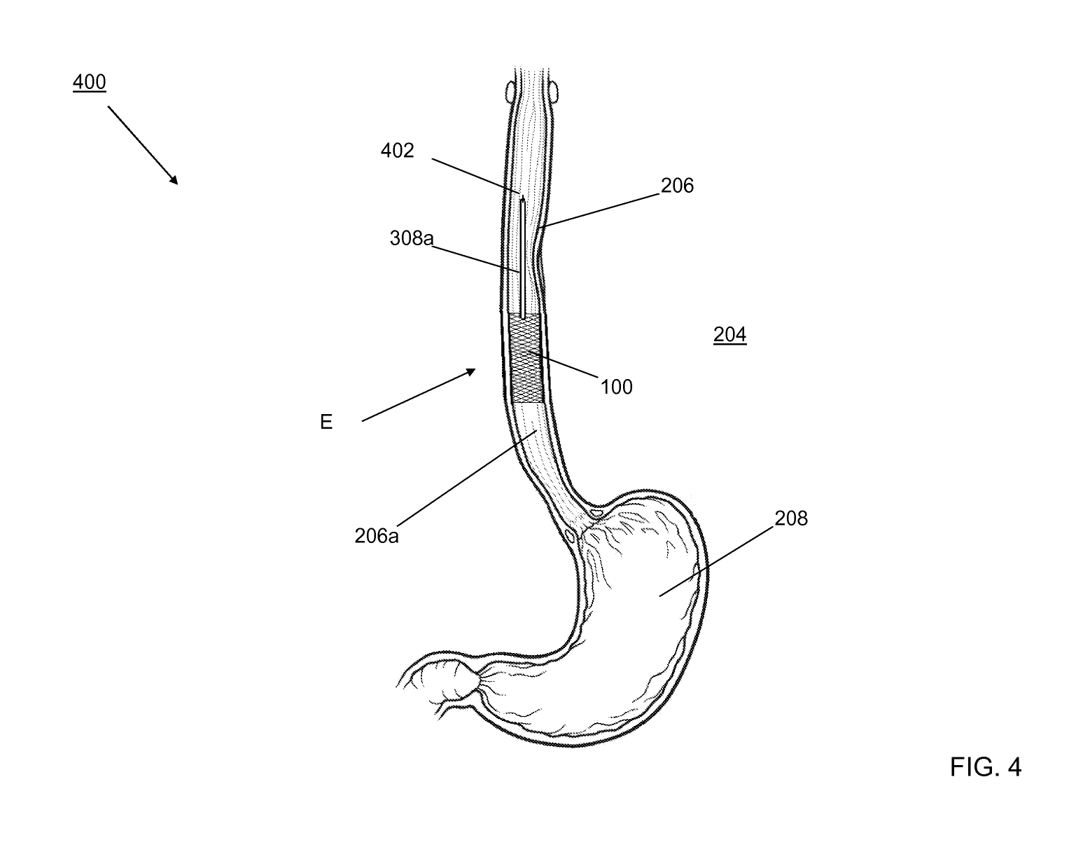

Referring now to FIG. 4, an alternative embodiment of the stent system and its operation according to the method 300 is illustrated. The stent system 400 is substantially similar in structure and operation to the stent system according to the method 300 discussed above with reference to FIG. 1, FIG. 2, and FIG. 3A-FIG. 3J, but with the provision of a modified block 312 and the removal of the one-way valve 112 from the stent 100. In an embodiment, at block 312, the migration-prevention bridle may be secured to a portion of the patient's body other than the nose 202a of the patient 200. For example, after the migration-prevention bridle 308b has been secured to the stent 100 at block 310 by threading the migration-prevention bridle 308b through the stent 100, an end or ends of the migration-prevention bridle 308b may be secured to the esophagus 206 by placing a staple 402 or other fixation device through the end or ends of the migration-prevention bridle 308b and the esophagus 206, as illustrated in FIG. 4. In other examples, securing the migration-prevention bridle 308b includes gluing the migration-prevention bridle 308b to the body part, suturing the migration-prevention bridle 308b to the body part, tying the migration-prevention bridle 308b to the body part, and/or a variety of other securing techniques known in the art. In an embodiment, the staple 402 or other fixation device is an absorbable staple or fixation device. Similarly as discussed above, with the migration-prevention bridle 308b secured to the stent 100 and the esophagus 206 through the staple 402, migration of the stent 100 is inhibited or preferably prevented. While examples of securing the migration-prevention bridle 308b to the nose 202a and esophagus 206 of the patient have been illustrated and described above, one of skill in the art will recognize that securing of the migration-prevention bridle 308b to a variety of other portions of the body of the patient 200 will fall within the scope of the present disclosure. For example, with the migration-prevention bridle 308b secured to the stent 100, the migration prevention-bridle 308a may also be secured to a nasogastric tube or the nasal septum. If the bridle cannot be secured to either of these, then an endoscopic clip (resolution clip) or T-fastener or other fixation device can be secured to the bridle to prevent distal migration. Furthermore, while the stent 100 is illustrated in FIG. 4 with the one-way valve 112 removed, in some embodiments, the one-way valve 112 may be included on the stent 100 of FIG. 4.

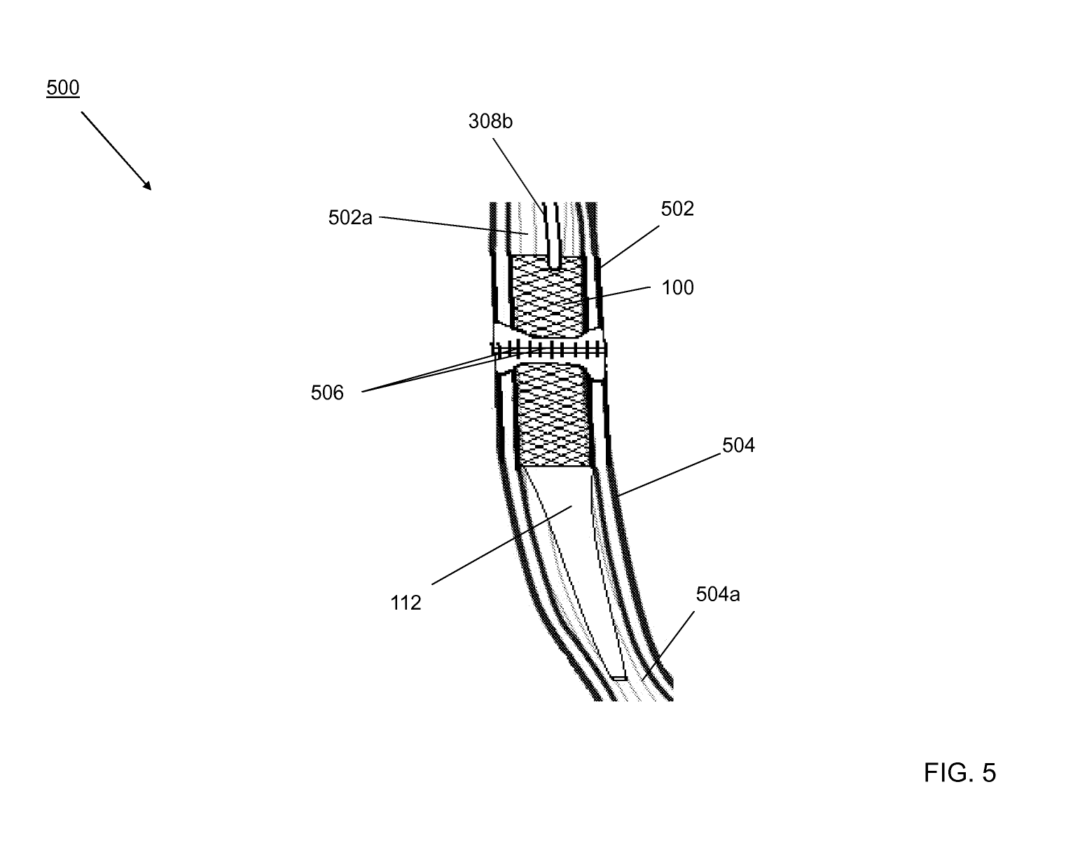

Referring now to FIG. 5, an alternative embodiment of the stent system 500 and its operation according to the method 300 is illustrated. The stent system 400 is at least substantially similar in structure and operation to the stent system according to the method 300 discussed above with reference to FIG. 1, FIG. 2, and FIG. 3A-FIG. 3J, but is used to assist in the healing of an anastomosis. FIG. 5 includes a first body part 502 defining a first body passageway 502a, and a second body part 504 defining a second body passageway 504a, each of which exist in the body of the patient 200, discussed above with reference to FIG. 2. In an embodiment, the first body part 502 and the second body part 504 may be portions of the bowel. As can be seen, a plurality of sutures 506 has been provided to connect the first body part 502 to the second body part 504. The stent 100 may be positioned in each of the first body passageway 502a and the second body passageway 504a according to the method 300 and expanded adjacent to the sutures 506 to assist in the healing of the anastomosis, with the migration-prevention bridle 308a secured and the one-way valve 112 operating as discussed in detail above.

For example, two ends of a bowel may be partially anastomosed posteriorly with an absorbable connector (or, alternatively, hand-sewn or otherwise secured) with the anterior portion of the anastomosis open. A guide wire may be placed in the distal bowel, and the stent may be guided over the guide wire to the location of the anastomosis. The stent may then be partially deployed and partially secured to a distal conduit that will be attached to the initially disconnected portion of the bowel. Once the distal end of the stent is fixed to the distal portion of the bowel downstream of the anastomosis, the proximal portion of the stent may be deployed. Securing of this proximal portion of the stent is optional. At this point, the anterior portion of the anastomosis is complete, providing a tension-free anastomosis that is end-to-end. Thus, the systems and methods discussed above may be used for anastomotic stenting. As can be appreciated from FIG. 5, a stent may be used to facilitate the creation of a tension-free anastomosis while protecting against leakage and allowing early enteral nutrition while the patient heals. In other embodiments, the systems and methods discussed above may be used for stenting to treat acute intrathoracic anastomotic leak after esophagectomy. The stent may be used to deploy into the esophagus or stomach to treat the leak or prophylactically prevent a leak. Anastomotic stenting is described as using the stent to both take the tension off of the anastomosis and then to buttress and cover the mucosa while the anastomosis heals. The intent would be for the stent to eventually be removed or degradable such that one healed, a stent is no longer needed or present within the lumen of the anastomosis.

In further embodiments, the apparatus, systems, methods, and kit discussed herein may also be used to treat anastomotic complications after bariatric surgery, gastric leaks such as for example, gastrojejunal anastomotic leaks after gastric bypass surgery, biliary abnormalities, vascular abnormalities, gastric abnormalities, and the like. Stenting during bariatric surgery would embody stenting from the esophagus to stomach or jejunum. The leakage of content is prevented both at the proximal and distal ends and concomitantly allows healing during oral nutrition. The radial force maintains the stent open but is monitored such that the anastomosis does not have enough radial force to prevent healing. Gastrojejunal leaks and fistulae may be either a part of bariatric surgery (where the stent is placed within a gastric pouch connected to jejunum roux en y, or to an entire stomach anastomosed to small bowel. Such leaks would also possibly require operative or percutaneous drainage, but the stent would allow passage of enteral alimentation during healing with minimal risk of reflux. The stent would be placed in a similar manner described for other enteral placement. Upon gaining access to the biliary tree, the stent in a smaller form could be used to treat leaks from biliary surgery including but not limited to hepatico-jejunostomy, biliary anastomoses to small bowel, or leaks within the biliary tree. Aortic aneurysms, large vessel disease with leaks or pseudoaneurysms, and anastomoses between vessels can also be healed or treated with the stenting mechanisms described above. Deployment would be similar to the GI tract. Patients with sleeve gastrectomy or gastric resections are also similarly benefactors of protection during healing of an anastomosis or a leak using this stenting technique.

It is understood that variations may be made in the above without departing from the scope of the invention. While specific embodiments have been shown and described, modifications can be made by one of ordinary skill in the art without departing from the spirit or teaching of this invention. The embodiments as described are exemplary only and are not limiting. Many variations and modifications are possible and are within the scope of the invention. Furthermore, one or more elements of the exemplary embodiments may be omitted, combined with, or substituted for, in whole or in part, with one or more elements of one or more of the other exemplary embodiments. Accordingly, the scope of protection is not limited to the embodiments described, but is only limited by the claims that follow, the scope of which shall include all equivalents of the subject matter of the claims.

What is claimed is:

1. A method for stent treatment, comprising:
positioning a stent adjacent a stent-repairable abnormality in a body passageway, wherein the stent includes a one-way valve;
securing a migration-prevention bridle that extends from the stent to a portion of the body;
resisting or preventing movement of the stent relative to the body passageway using the migration-prevention bridle; and
inhibiting or preventing passage of material through the body passageway in one direction using the one-way valve.

2. The method of claim 1, which further comprises: threading the migration-prevention bridle through an opening in the stent such that the migration-prevention bridle extends from the stent, wherein the threading is performed subsequent to the positioning of the stent in the body passageway adjacent the stent-repairable abnormality.

3. The method of claim 1, wherein a retrieval loop is located on an end portion of the stent, and wherein the opening in the stent to which the migration-prevention bridle is threaded is spaced apart from the retrieval loop.

4. The method of claim 1, wherein the portion of the body to which the migration-prevention bridle is secured includes a nose, and wherein the method further comprises:
extending a first portion of the migration-prevention bridle through a first nostril defined by the nose;
extending a second portion of the migration-prevention bridle through a second nostril defined by the nose; and
securing each of the first and second portions of the migration-prevention bridle to each other to secure the migration-prevention bridle to the nose.

5. The method of claim 1, wherein the portion of the body to which the migration-prevention bridle is secured includes the body part, and wherein the securing includes one of stapling the migration-prevention bridle to the body part, gluing the migration-prevention bridle to the body part, suturing the migration-prevention bridle to the body part, and tying the migration-prevention bridle to the body part.

6. The method of claim 1, further comprising: disconnecting the migration-prevention bridle from the stent.

7. The method of claim 6, further comprising: removing the stent from the body passageway using a retrieval loop that is secured to an end portion of the stent and that is adapted to reduce the diameter of the end portion of the stent.

8. A method for repairing an esophageal perforation in a patient, comprising:
positioning a stent in an unexpanded orientation in an esophageal passageway defined by an esophagus of a patient, wherein the stent includes a one-way valve;
expanding the stent to an expanded orientation after the stent is located adjacent an esophageal perforation defined by the esophagus;
extending a migration-prevention bridle into the esophageal passageway such that a first portion of the migration-prevention bridle is located adjacent the stent;
securing the first portion of the migration-prevention bridle to the stent;
securing a second portion of the migration-prevention bridle to a nose of the patient; and
inhibiting or preventing migration of the stent using the migration-prevention bridle, wherein the flow of material through the esophageal passageway is controlled using the one-way valve.

9. The method of claim 8, further comprising:
threading the second portion of the migration-prevention bridle through an opening in the stent to secure the migration-prevention bridle thereto.

10. The method of claim 8, further comprising:
extending a first portion of the migration-prevention bridle through a first nostril defined by the nose;
extending a second portion of the migration-prevention bridle through a second nostril defined by the nose; and
securing the first and second portions of the migration-prevention bridle to each other to secure the migration-prevention bridle to the nose.

11. The method of claim 8, further comprising:
releasing the first portion of the migration-prevention bridle from the stent and removing the migration-prevention bridle from the esophageal passageway.

12. The method of claim 8, which further comprises determining if a desired amount of tissue ingrowth to the stent has occurred before the unsecuring and removing.

13. The method of claim 8, further comprising:
removing the stent from the body passageway using a retrieval loop operatively coupled to the stent to reduce a diameter of at least a portion of the stent.

14. A method to prevent stent migration, which comprises:
positioning a stent in a body passageway in a patient's body such that the stent is located adjacent a stent-repairable abnormality;
securing a migration-prevention bridle that extends from the stent to body passageway by connecting a fixation device to the migration-prevention bridle and the body passageway; and
resisting or preventing movement of the stent relative to the body passageway using the migration-prevention bridle.

15. The method of claim 14, wherein the body passageway is an esophagus.

16. The method of claim 14, wherein the fixation device is a staple.

17. A method to repair an anastomosis of the bowel in a patient, which comprises:
positioning a stent in an unexpanded orientation in an bowel passageway defined by a bowel of a patient, wherein the stent includes a one-way valve;
expanding the stent to an expanded orientation after the stent is located adjacent the anastomosis defined by the bowel;
extending a migration-prevention bridle into the bowel passageway such that a first portion of the migration-prevention bridle is located adjacent the stent;
securing the first portion of the migration-prevention bridle to the stent;
securing a second portion of the migration-prevention bridle to a body part of the patient; and
resisting or preventing migration of the stent using the migration-prevention bridle;
wherein the flow of material through the esophageal passageway is controlled with the one-way valve.

18. A method to repair an abnormality in a gastrointestinal system of a patient, which comprises:
positioning a stent in an unexpanded orientation in a passageway defined by the gastrointestinal system of the patient, wherein the stent includes a one-way valve;
expanding the stent to an expanded orientation after the stent is located adjacent an abnormality in the gastrointestinal system;
extending a migration-prevention bridle into the passageway such that a first portion of the migration-prevention bridle is located adjacent the stent;
securing the first portion of the migration-prevention bridle to the stent;
securing a second portion of the migration-prevention bridle to a body part of the patient; and
resisting or preventing migration of the stent using the migration-prevention bridle, wherein the flow of material through the esophageal passageway is controlled by the one-way valve.

19. The method of claim 18, wherein the gastrointestinal system is at least one of an esophagus, a stomach, a small bowel, a large bowel, and a rectum.

20. The method of claim 18, wherein the abnormality includes one or more of an anastomotic leak, a stricture, an injury, an aortoesophageal fistula, a spontaneous perforation, an intrathoracic leak, a leak associated with morbid obesity surgery, an esophageal dilatation, a non-perforating lesion, dysphagia, cancer, and any combination thereof.
